(19) United States
(12) Patent Application Publication
(10) Pub. No.: US 2018/0008452 A1
(43) Pub. Date: Jan. 11, 2018

(54) Mouth Closing Device

(71) Applicant: Richard E. Wade, Leesburg, IN (US)

(72) Inventor: Richard E. Wade, Leesburg, IN (US)

(21) Appl. No.: 15/640,788

(22) Filed: Jul. 3, 2017

Related U.S. Application Data
(60) Provisional application No. 62/358,196, filed on Jul. 5, 2016.

(51) Int. Cl. A61F 5/56; A61M 16/00; A61M 16/06

(52) U.S. Cl. CPC A61F 5/56; A61M 16/0683; A61M 16/0057; A61M 16/0666; A61M 2209/088

(57) ABSTRACT

The present invention relates to a device to urge the mouth closed during sleep to reduce snoring and/or treat sleep disorders such as sleep apnea. In one aspect, the device includes a jaw-blocking object that is secured beneath the chin of a user and also contacting the clavicular region of the upper chest area. In another aspect, a device of the invention includes a jaw-blocking object in combination with a CPAP mask and headgear for use with a CPAP system.

### CROSS REFERENCE TO RELATED APPLICATIONS

This application claims the benefit of U.S. Provisional Application No. 62/358,196 filed Jul. 5, 2016, the entire contents of which are herein incorporated by reference.

### FIELD OF THE INVENTION

The present invention relates to a device and methods for reducing snoring and treating sleep apnea, more specifically to an improved device for more effectively holding the jaw closed during sleep.

### BACKGROUND OF THE INVENTION

Sleep related breathing disorders such as sleep apnea and snoring are fairly common in the general population. By some estimates more than 18 million adult Americans and children suffer from sleep apnea, with only 10% of them being diagnosed. The most common type of sleep apnea is obstructive sleep apnea (OSA) in which obstructions in the airway cause breathing to stop during sleep. Another type of sleep apnea, central sleep apnea (CSA), is caused by a defect in the brain or heart and is unrelated to blockage of the airway. Sleep apnea can affect adults, children, and infants.

A characteristic feature of sleep apnea is repeated bouts of breathing stops during the night. The number of breathing stops can exceed 100 in some individuals. In many instances, the brain awakens the patient to resume breathing. However, when this does not occur a patient's condition can become critical. While surgery is sometimes recommended for treating sleep apnea, the most common type of treatment involves use of a CPAP (Continuous Positive Airway Pressure) system in which an air flow generator pushes air through a mask into the patient's airway to keep it open during sleep.

While CPAP therapy provides considerable benefit, including improved breathing and oxygenation, noncompliance by patients is a major concern for medical professionals and insurance carriers. Persistent problems arising with CPAP therapy include air leakage, pain, discomfort, and a constricted airway. When the mouth partially opens during treatment with a nasal mask, CPAP treatment can become ineffective, and gastric insufflation may result as the airway becomes constricted. As a consequence of these and other problems, some patients may elect to avoid CPAP therapy entirely, or use it only sporadically.

A number of chin straps and other mouth-closing devices are known for use in urging the mouth closed during sleep. For example, U.S. Pat. No. 5,289,829 provides a flexible, bent tubular U-shaped device having an arcuate bottom that engages with the upper chest of a user and two upwardly extending J-shaped arms with short extensions that make contact with the facial region above the jaw line on both sides of the chin to maintain the lips in a closed position.

U.S. Pat. No. 5,787,894 provides an anti-snoring device having two upwardly extending straps around the lower jaw that encircle the head. In another embodiment, the device provides a chin cup on one end of a strap to receive a user's chin.

U.S. Pat. No. 8,701,655 provides an H-shaped chin strap assembly having a central region that cups around a user's chin for use with a respiratory machine.

U.S. Pat. Appn. Pub. No. 2014/0360505 provides a chin cup and strap assembly for use with a CPAP system. The device provides a restraining force against the chin by means of the chin cup and strap assembly without any additional impediment to mouth opening or forward head tilt.

While prior devices provide some useful features, they are not fully satisfactory. Chin straps allow the mouth to partially open, and if secured too tightly can be uncomfortable to wear. There remains a need for an improved mouth-closing device for stand-alone use or use with a CPAP machine to reduce snoring and/or treat sleep disorders such as sleep apnea.

### SUMMARY OF THE INVENTION

This Summary is provided merely to introduce certain concepts and is not intended to identify any key or essential features of the claimed subject matter. Certain embodiments of the invention relate to an improved mouth-closing device that can be used alone or in combination with a CPAP system.

In one aspect, a device of the invention provides a jaw blocking object which is placed between the underside of the chin and the suprasternal notch region of a user's upper chest to block the jaw from opening for use in reducing snoring and/or treating sleep disorders such as sleep apnea.

In another aspect, a device of the invention includes a jaw-blocking object and harness assembly to secure the jaw-blocking object to a user's head, or alternatively to a CPAP mask and/or CPAP headgear when using the device with a CPAP system.

In another aspect, a device of the invention can be used to comfortably and efficiently urge a user's jaw to a closed position without inducing undue stress on the mandible, a feature especially beneficial for sleep apnea patients who concomitantly suffer from TMD (Temporomandibular Joint Dysfunction).

In another aspect, a device of the invention is used to reduce air swallowing and gastric insufflation when used with a CPAP system by preventing a user's head from rolling forward, thereby promoting an open airway.

In another aspect, the invention relates to a therapeutic method to reduce or eliminate snoring and/or to treat sleep apnea in sufferers of any age including premature infants, children and adults, by wearing a device of the invention alone or in combination with a CPAP system.

These and other aspects of the present invention will be apparent from the following description, the accompanying drawings, and the appended claims.

### BRIEF DESCRIPTION OF THE DRAWINGS

FIG. 1 shows a prior art chin strap.

FIG. 2A provides a schematic side view showing the MNC region of an individual between the mandible and upper chest area.

FIG. 2B provides a front view of the MNC region between the mandible and suprasternal notch region at the upper chest area of an individual.

FIG. 3A shows a perspective view of a spherically-shaped jaw stop of a device of the invention.

FIG. 3B shows a side view of the jaw stop of FIG. 3A placed in the MNC region of a wearer.

FIG. 3C shows a front view of the jaw stop of FIG. 3A placed in the MNC region of a wearer.

FIG. 3D shows a front perspective view of the jaw stop in FIG. 3C after lateral head rotation.

FIG. 4A shows a perspective view of an embodiment of a hollow jaw stop of the invention having a spherical or ball shape.

FIG. 4B shows a cross-sectional view of the jaw stop of FIG. 4A taken at line 4B-4B.

FIG. 4C shows a perspective view of an embodiment of a jaw stop of the invention having a oblate spheroid shape.

FIG. 4D shows a perspective view of an embodiment of a jaw stop of the invention having a prolate spheroid shape.

FIG. 4E shows a perspective view of an embodiment of a solid jaw stop of the invention having a hemispherical shape.

FIG. 4F shows a perspective view of the embodiment of a jaw stop depicted in FIG. 4E having a chin notch section removed from the top surface.

FIG. 4G shows a perspective view of an embodiment of a jaw stop of the invention having a cuboid shape.

FIG. 4H shows a perspective view of an embodiment of a jaw stop of the invention having a cylindrical shape.

FIG. 4I shows a perspective view of an embodiment of a jaw stop of the invention having a parabolic shape.

FIG. 4J shows a perspective view of the embodiment of a jaw stop depicted in FIG. 4I having a chin notch on the top side.

FIG. 5A shows a front perspective exploded view of an embodiment of a device of the invention including harness assembly.

FIG. 5B shows a front perspective view of an embodiment of a device of the invention showing a jaw stop mounted in a harness assembly.

FIG. 6A shows a side view of a device of the invention including one embodiment of a harness assembly when worn by a user without a CPAP system.

FIG. 6B shows a side view of a device of the invention including another embodiment of a harness assembly when worn by a user without a CPAP system.

FIG. 7A shows a front view of an embodiment of a device of the invention secured to the harness assembly of a CPAP nasal mask on a user.

FIG. 7B shows a side view of an embodiment of a device of the invention secured to the harness assembly of a CPAP nasal mask on a user.

### DETAILED DESCRIPTION OF PREFERRED EMBODIMENTS

As used herein the term “CPAP” refers to Continuous Positive Airway Pressure.

As used herein the term “CPAP system” refers to a breathing machine, a facial mask, and headgear through which continuous positive airway pressure is provided to a patient suffering from a sleep disorder such as sleep apnea.

As used herein, the term “CPAP mask” refers to a mask worn when using a CPAP system including any number of known and/or commercially available devices including nasal pillows, nasal masks, full-face masks, nasal prongs, hybrids, oral devices, and total face masks. A CPAP mask is generally secured to a user's head with a strap assembly or a CPAP headgear.

As used herein, the term “harness assembly” refers to one or a plurality of straps, preferably adjustable, adapted for securing a jaw-stop member of the invention directly to a user's head, or to a CPAP mask and/or headgear.

As used herein, the terms “jaw stop” or “jaw block” or “jaw blocking object” are used interchangeably to refer to an aspect of a device of the invention. A jaw stop is a solid or hollow object having a volume, of any size and geometric shape other than bent tubular, adapted to generally occupy the MNC region beneath a user's chin to act as an impediment to jaw opening. The jaw stop makes contact beneath a user's chin and at the upper chest area thereby blocking movement of the jaw while keeping the head substantially vertically aligned with the neck and upper spine. A jaw stop of the invention does not make contact with a user's face above the jaw line or with the facial flesh on the sides of the chin.

As used herein the term “sleep apnea” refers to any sleep disorder characterized by bouts of breathing stops during sleep, including obstructive sleep apnea (OSA) and central sleep apnea (CSA). The terms may also include other conditions sometimes associated with sleep apnea such as temporomandibular joint dysfunction (TMJ).

As used herein, the term “mandible neck curve” or “MNC” is used to describe the curvilinear space into which a jaw stop of the invention is placed. The MNC begins at the chin on the underside of the jaw, and proceeds generally along the midline down the front side of the neck to the suprasternal notch region at the upper chest. A jaw stop of the invention is placed in the MNC to block or prevent the chin from dropping, thereby urging the mouth to a closed position. The terms “upper MNC” or “upper region of the MNC” may sometimes be used to refer specifically to the region beneath the chin, and “lower MNC” or “lower region of the MNC” may sometimes be used to refer specifically to the region at or near the suprasternal notch.

As used herein, the term “treat” or “treatment” refers to application of a device of the invention to reduce or eliminate snoring, breathing stops, mouth-breathing, and air swallowing, as well as to maintain an open airway, when used with or without a CPAP system, in treating sleep disorders including sleep apnea.

As used herein, the term “suprasternal notch” or “suprasternal notch region” refers to the region at the base of the neck characterized by a visible depression above the clavicle and collar bone. A portion of a jaw stop of the invention preferably makes contact in this region, or the region immediately surrounding the suprasternal notch and clavicle.

Without intending to be bound by any particular theory, it is believed that a jaw-blocking device of the invention improves on prior mouth-closing devices such as chin straps by providing an obstructive object directly beneath the chin to more effectively urge the mouth closed during sleep while at the same time keeping the head from rolling forward. Prior devices such as chin straps rely solely on a cup and strap assembly to apply a pulling force against the chin. Such devices are often ineffective because the force generated by the cup and strap assembly includes a posterior-directed component that tends to pull the jaw inward (i.e. in a posterior direction), thereby encouraging the mouth to open and defeating the intended purpose.

By contrast to the prior art, a device of the present invention includes a jaw-blocking object placed directly beneath the chin and generally occupying a user's MNC region without contacting the facial region above the jaw line. The jaw blocking object provides a substantially vertical force against the underside of the chin region, and an additional generally anterior force generated at the suprasternal notch contact region of the jaw stop. As a consequence of these combined force vectors, a device of the invention is more effective in urging the mouth to a closed position, and in preventing the head from rotating forward, thereby further promoting an open airway. With the jaw urged to a closed position, air leakage is reduced during CPAP treatment, the airway remains open, and gastric insufflation is reduced or eliminated.

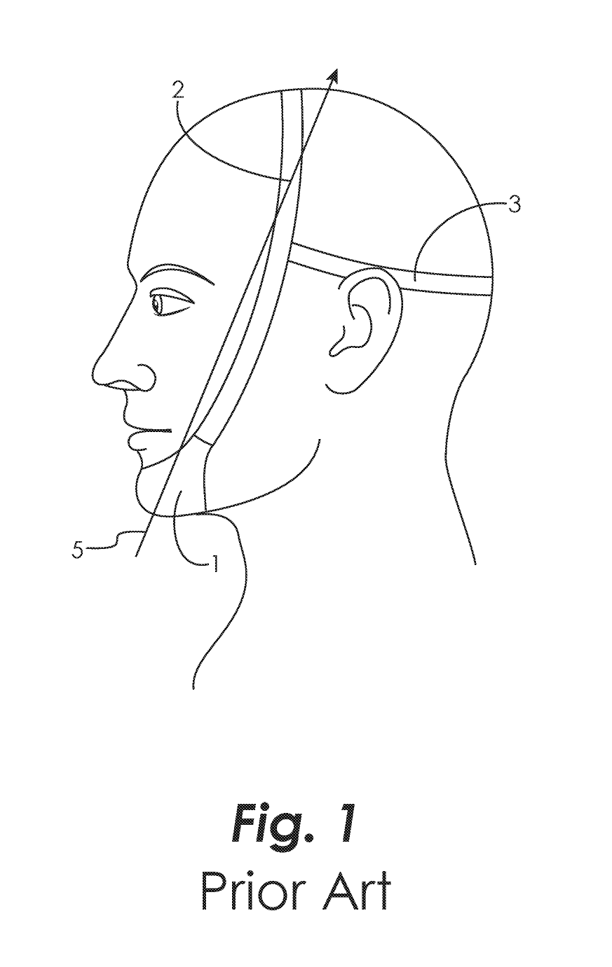

Referring now to the drawings, preferred embodiments of the invention will now be described in more detail. For purposes of comparison with the prior art, FIG. 1 shows a prior art chin strap which includes a chin cup 1 to receive a user's chin, an overhead strap 2 secured to the chin cup, and an around-the-head strap 3 that is joined to the overhead strap. A force vector 5 generated by the cup and strap assembly includes a posterior-directed force component that tends to pull the jaw backward, thereby encouraging the mouth to open and undermining the intended purpose.

A device of the invention includes a jaw-blocking object, also referred to herein as a “jaw stop”, which urges the jaw to a closed position by being an impediment within the MNC region to block jaw movement, and by applying a vertically directed force from beneath the chin and from the upper chest at the clavicular region. In addition to urging the mouth closed, an additional benefit of a device of the invention is that it prevents the head from rotating forward, thereby keeping the airway open. A jaw stop can be of any size and/or geometric shape other than bent tubular to comfortably fit in the MNC region of a user to impede jaw opening and forward head rotation, having a symmetrical or asymmetrical configuration, and one or more convex or concave surfaces. A jaw stop can be solid or have a hollow interior.

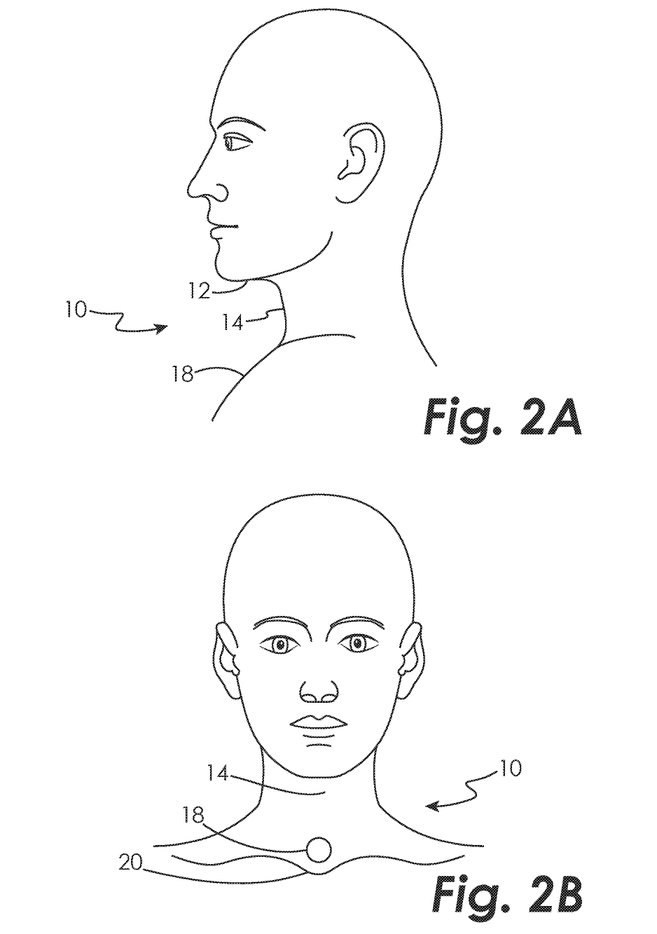

FIGS. 2A-2B illustrate the position of the MNC region on a user. The MNC region 10 correlates with the space under the midline of the chin 12, proceeding around the front of the neck 14, and down to the upper chest area at the suprasternal notch 18 adjacent the clavicle 20.

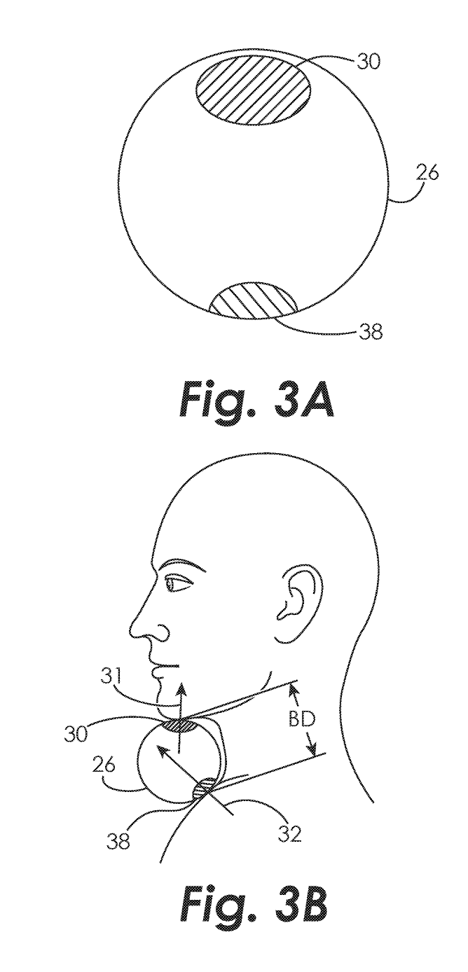

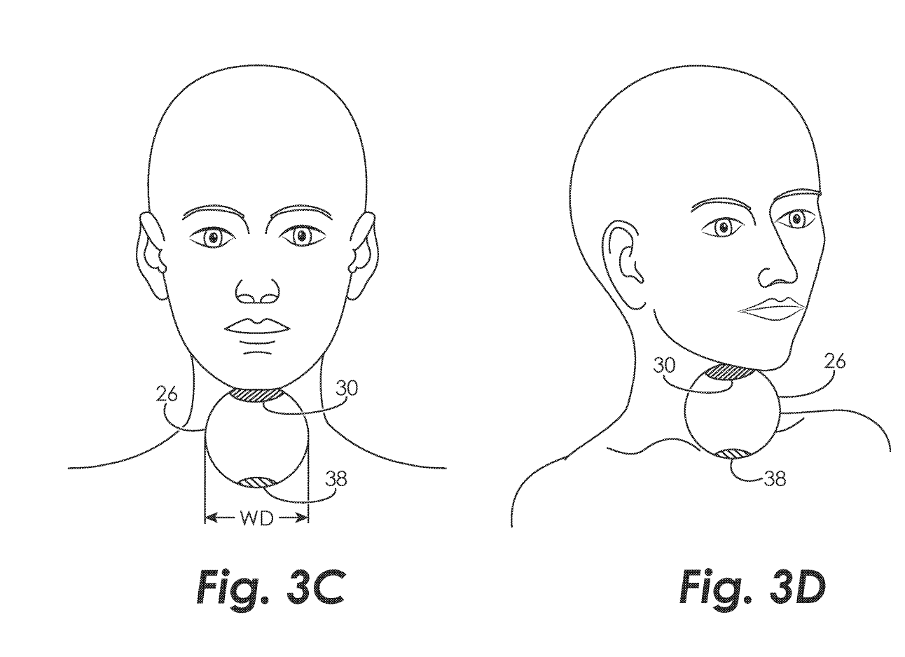

Referring now to FIGS. 3A-3D, certain aspects of a jaw stop of the invention are illustrated. FIG. 3A shows a preferred embodiment of a jaw stop 26 having a spherical shape. The jaw stop provides two contact regions on the surface that make contact with a user in the MNC region. The contact regions can be contiguous or non-contiguous on the surface of the jaw stop. First contact region 30 makes contact at least along the midline under the chin, while second contact region 38 makes contact at or near the suprasternal notch. FIG. 3B shows placement of jaw stop 26 in the MNC space of a user. First contact region 30 generates a substantially vertical or slightly posterior force vector 31 beneath the chin, while second contact region 38 generates a substantially anterior force vector 32 directed away from the suprastemal notch region. Depending on the size and shape of the jaw stop, the surface thereof may contact other portions of the MNC such as the frontal region of the neck. Unlike prior chin straps, the combined forces from beneath the chin 31 and the suprastemal notch region 32 do not pull back on the jaw but instead exert a substantially vertically directed force, thereby more effectively urging the jaw to remain closed.

FIGS. 3C and 3D further illustrate the jaw stop 26 placed in the MNC region showing that the jaw is substantially prevented from opening while also impeding forward rotation of the head. This helps in keeping the airway open. In addition, the configuration of the jaw stop and contact regions generally allow some side-to-side head movement to afford additional comfort during use (FIG. 3D).

A jaw stop of the invention can have any geometric shape or configuration other than bent tubular including but not limited to cuboid, cylindrical, spherical, hemispherical, ellipsoid, prolate spheroid, oblate spheroid, polygonal spheroid, and any other polygonal shape. Preferably the shape is symmetrical and has curved surfaces. The most preferred shape is spherical. When placed in the MNC region during use, the jaw stop remains below the jaw line.

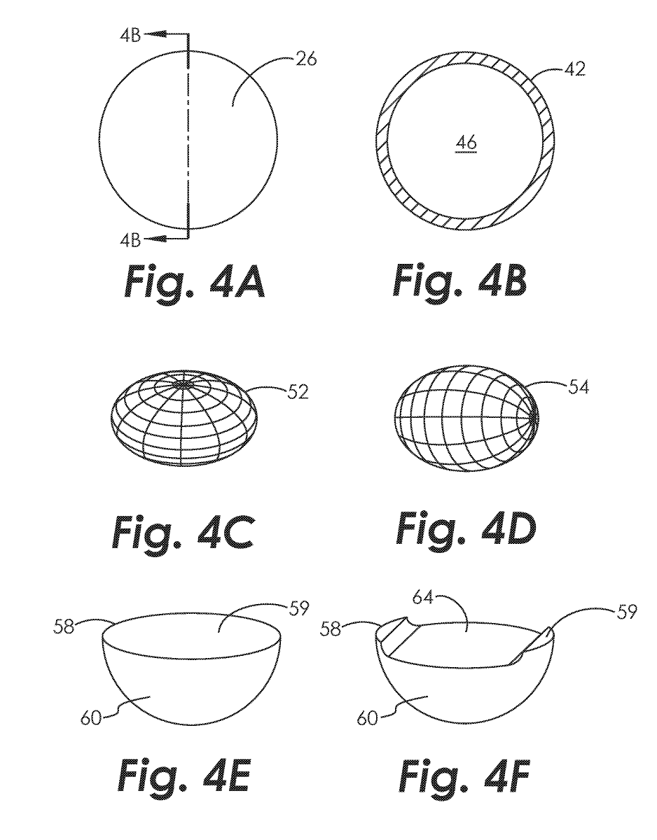

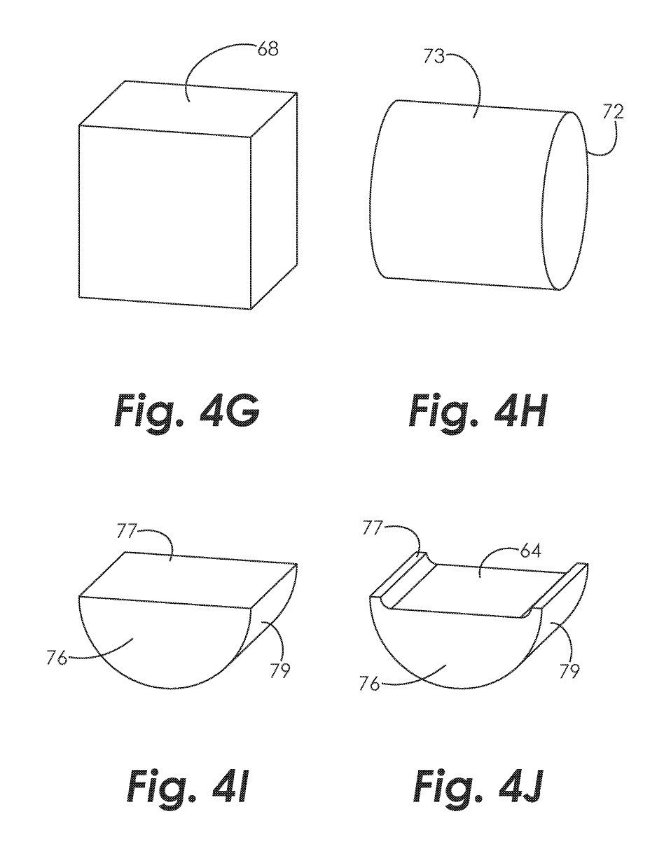

Exemplary but non-exclusive jaw stops having different shapes are presented in FIGS. 4A-4J. FIG. 4A illustrates a spherical, ball-shaped jaw stop 26, and FIG. 4B shows a cross-section view along line 4B-4B showing a hollow interior 46 and surrounding wall 42. FIGS. 4C-4D illustrate an oblate-spheroid jaw stop 52, and a prolate-spheroid jaw stop 54, respectively. FIG. 4E illustrates a hemispherical jaw stop 58 having a flat top region 59 and rounded bottom 60. FIG. 4F illustrates a hemisphere-shaped jaw stop 58 having a chin notch 64 on the flat top 59. FIG. 4G illustrates a cuboid-shaped jaw stop 68. FIG. 4H illustrates a cylindrical-shaped jaw stop 72 having curved surface 73. FIG. 4I illustrates a jaw stop having a cylindrical half-section shape 76, flat top 77, and curved bottom 79. FIG. 4J shows the half cylindrical jaw stop 76 of FIG. 4I with a chin notch 64 on the flat top portion 77.

Particularly preferred is a sphere-shaped or ball-shaped jaw stop which comports well with the concave curvature of the MNC region. Additionally, the arcuate surface of a sphere is well adapted to allow for some side-to-side head rotation which provides additional comfort during use.

As provided in Table 1, depending on the shape of a jaw-blocking object, the contact regions can be located anywhere on the surface of the object, or at certain defined regions. For spherically-shaped objects, owing to the symmetry, the contact regions are generally located anywhere on the curved surface. For other shapes such as those illustrated in FIGS. 4E-4J, the contact regions are at fixed locations on the surface. For example, the suprasternal notch contact region is located at the rounded bottom portion such as those in FIGS. 4I and 4J, while the under chin-contact region is located elsewhere, for example at a flat or notched top portion as in FIGS. 4F and 4J.

TABLE 1
Exemplary jaw blocking objects and location of contact regions.

| | | Location of Contact Region | |
|---|---|---|---|
| Jaw Stop Shape | Figure | Under-chin | Suprasternal notch |
| Sphere | 4A | Anywhere on surface | Anywhere on surface |
| Prolate sphere | 4C | On curved surface | On curved surface |
| | | between ends defined | between ends defined |
| | | by axis of rotation | by axis of rotation |
| Oblate sphere | 4D | On curved surface | On curved surface |
| | | between ends defined | between ends defined |
| | | by axis of rotation | by axis of rotation |
| Hemisphere | 4E | Flat face | Rounded bottom |
| Cuboid | 4G | Flat face | Flat face |
| Cylinder | 4H | Curved surface | Curved surface |
| Sectional cylinder | 4I | Flat top | Curved bottom |
| Sectional cylinder | 4J | Chin notch | Curved bottom |
| with chin notch | | | |

In some embodiments of a jaw blocking object, one or more of the contact region(s) have a curved surface, preferably convex, which facilitates rolling and side-to-side head movement when the device is worn.

A jaw stop can be any size that comfortably fits in the MNC region beneath the jaw to urge the mouth closed. Generally, length, width, and depth dimensions would be sized smaller to fit children and larger to fit adults. By way of example, for a spherically-shaped jaw stop, an appropriate diameter would generally be in a range of about 1 to about 5 inches, or larger including 1 inch, 2 inches, 3 inches, 4 inches, or 5 inches, and all decimal increments therebetween. Generally, for a small size, the diameter would be from about 1 to about 3 inches, including all whole and fractional integers therebetween; a medium size would be from about 3 to about 4 inches including all whole and fractional integers therebetween; and a large size would be from about 4 to about 5 inches or larger, including all whole and fractional integers therebetween. An appropriate size could be determined by measuring the distance from near the end of the chin of a user to the suprasternal notch (BD in FIG. 3B) and sizing the sphere or ball accordingly. An appropriate size could also be determined based on the approximate width span of a user's jaw (WD in FIG. 3C). For shapes other than a sphere or spheroid, length, width, and depth dimensions would be sized so that the device would comfortably fit in the MNC region of a user.

A jaw stop can have any desired level of hardness or softness and resistance to deformation. Preferably, the object provides enough resistance to act as an effective barrier to jaw opening while not causing discomfort to the wearer. A jaw stop may be stiff, rigid, or firm, or alternatively, soft and/or flexible or semi-flexible and resilient to deformation. A jaw stop can be fabricated by methods known to the skilled artisan, from any suitable material including but not limited to foam, such as polystyrene, plastic, polyethylene, hard or soft rubber, silicone, wood, metal, woven or nonwoven fabric, leather, or textile. In the preferred embodiment, a jaw stop is hollow and may have perforated walls to reduce weight and increase comfort. The jaw stop can optionally be covered with a soft and/or smooth material such as plastic, leather, neoprene, fabric or the like for added comfort against the skin.

In an alternative embodiment, a jaw stop comprises an inflatable shape fabricated from any suitable plastic or rubber material including but not limited to vinyl, latex, polyvinyl chloride (PVC), or neoprene, having a valve stem or other means for blowing air into the shape to inflate, or to release air to deflate the shape. An inflatable jaw stop can be fabricated to have any desired shape including, but not limited to, those depicted in FIGS. 4A-4J by methods known to the skilled artisan including, but not limited to, a dip molding process.

A mouth-closing device of the invention contemplates use of a jaw stop alone or in combination with a CPAP system. For use with a CPAP system, a jaw blocking device of the invention can be retrofitted to an existing CPAP mask and/or headgear, or alternatively integrated with a CPAP mask and headgear during manufacture of the CPAP equipment.

In another aspect, the invention relates to a harness assembly for securing a jaw stop to a wearer's head, or to a CPAP mask or CPAP headgear. Any suitable harness assembly or headgear known to the skilled artisan is contemplated for this purpose, including but not limited to, a strap or harness assembly having one or more straps that can be adjusted to fit around a user's head and that engage with the jaw stop. Generally, a harness assembly would bridge over and/or around the wearer's neck or head to secure the jaw stop beneath the chin. The harness assembly may be adapted to couple with a CPAP mask and/or CPAP headgear. A strap or harness assembly can engage with the jaw stop by any suitable means including, but not limited to, by securing one or more strap(s) of a harness or headgear to a portion(s) of the jaw stop, or alternatively to a pocket or other receptacle on the harness suitably configured to receive a jaw stop.

In one embodiment, a jaw stop is secured to a user's head for stand-alone use without a CPAP system. In another embodiment, a harness assembly is adapted to attach a jaw stop to a CPAP mask or CPAP mask and headgear for use with a CPAP system. Any known and/or commercially available CPAP mask and headgear can be used for this aspect of the invention including nasal pillows, nasal masks, full-face masks, nasal prongs, hybrids, oral devices, and total face masks, and harness assemblies therefor as disclosed, for example, in U.S. Pat. No. 8,701,655, U.S. Pat. No. 8,899,234, U.S. Pat. No. 6,470,886, US 2014/0360505, US 2007/0209663, and U.S. Pat. No. 7,036,508, the entire contents of which are herein incorporated by reference. Suitable commercially available CPAP products are widely available and sold by a number of companies including, for example, 3B Medical (Winterhaven, Fla.), AG Industries (St. Louis, Mo.), and ResMed (Australia). Exemplary but not exclusive CPAP masks include a number of nasal masks having 4-point headgear sold by ResMed such as Mirage SoftGel®, Mirage Quattro®, and Mirage Activa®. It should be understood that this aspect of the invention is intended to include any known and/or commercially available type of CPAP mask and/or headgear.

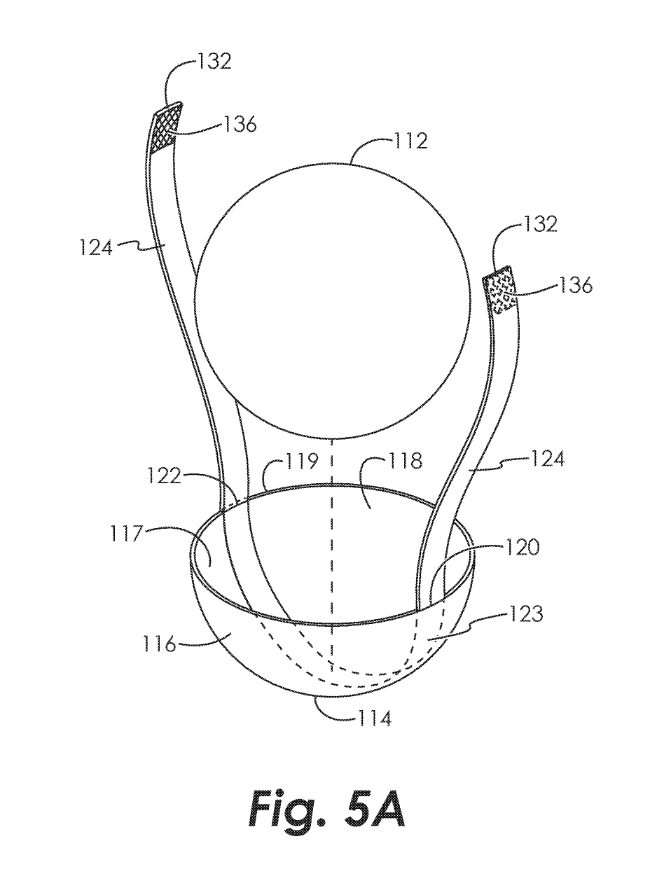

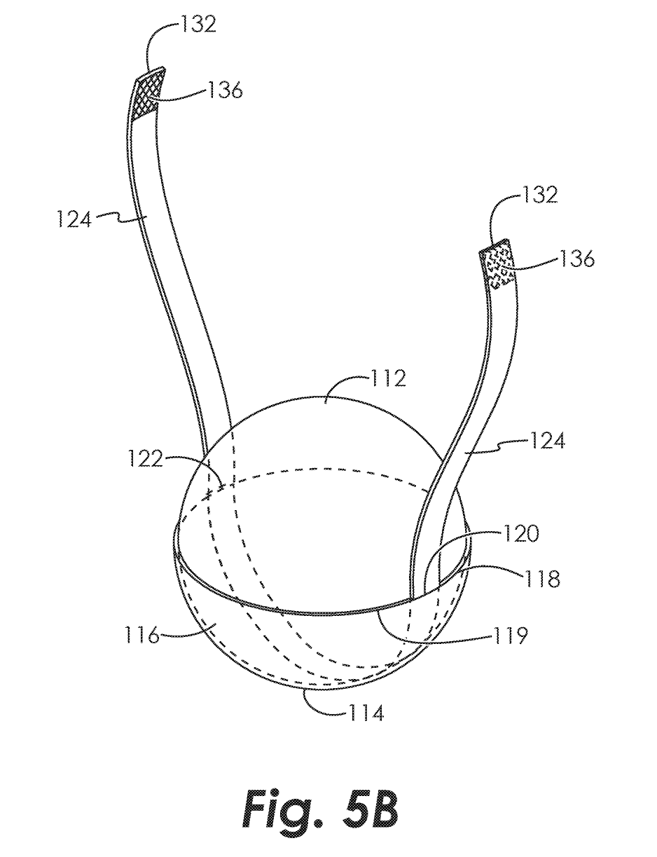

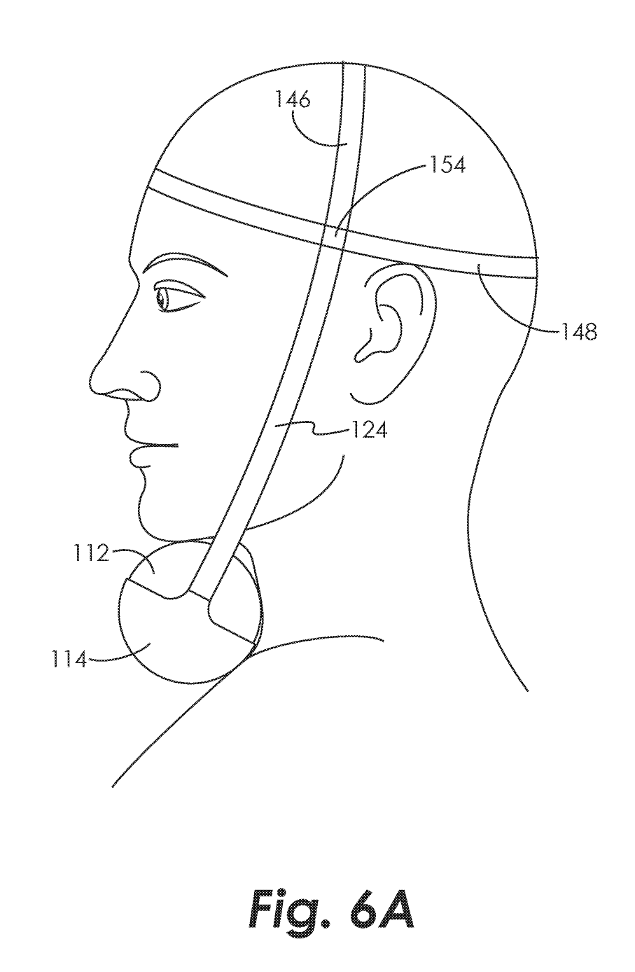

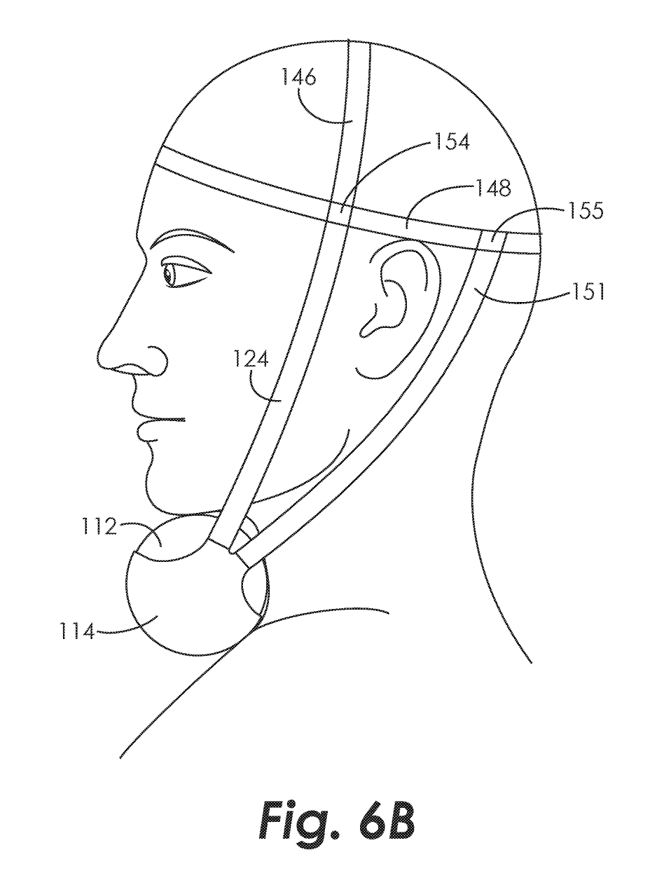

FIGS. 5A-5B and 6A-6B illustrate exemplary embodiments of a device of the invention adapted for use without a CPAP system (FIG. 6A-6B), or for use with a CPAP mask and headgear (FIGS. 5A-5B). While a spherically-shaped jaw stop is illustrated, it is to be understood that other shapes are intended to be within the scope of this aspect of the invention. FIGS. 5A-5B illustrate an embodiment for use with a CPAP mask and headgear or harness assembly. Jaw stop 112 engages with a receiving pocket 114 that is suitably configured to receive the jaw stop within cavity 117. Pocket 114 has a size and shape that is adapted to receive a portion or all of the jaw stop within cavity 117. In one embodiment, pocket 114 includes a front face 116, back face 118, top perimeter edge 119 that encircles jaw stop 112 when placed in pocket 114 (FIG. 5B), bottom portion 123, and opposing ends 120 and 122 at which first and second attachment straps 124 are joined. For attachment to a CPAP mask and/or headgear or harness assembly, attachment straps 124 include ends 132, having regions 136 for coupling to a CPAP mask and headgear or harness assembly. Preferably, regions 136 provide hook and loop fasteners (i.e. VELCRO® as shown in FIGS. 5A-5B) or snap fasteners that can be reversibly coupled to counterpart regions on a CPAP mask and headgear, or other harness assembly. The attachment straps can be fabricated from any suitable material including but not limited to fabric, neoprene, vinyl, plastic, or leather.

Pocket 114 can be fabricated from any suitable flexible, semi-rigid, or rigid material such as plastic, rubber, wood, leather, or fabric, by means known to the skilled artisan, as a single unit or by joining pieces together, for example, by adhesives, sewing, and the like.

In another aspect, a device of the invention is adapted to be used alone without a CPAP system. In this aspect, a jaw stop is secured to the head of a user with any type of harness assembly known to the skilled artisan. Referring now to FIGS. 6A-6B, embodiments according to this aspect of the invention include overhead strap 146 which is placed over the top of a user's head and joined on both sides to head strap 148 at junctions 154. A pair of forward straps 124 depend from junction 154 in front of a user's ears to connect with jaw stop pocket 114 (FIG. 6A). In an alternative embodiment (FIG. 6B), a pair of rearward straps 151 are included which depend behind the ear from junction 155 to connect with jaw stop pocket 114. Straps 124 and 151 are preferably reversibly connected to junction 154 and 155 by any suitable means including, but not limited to, snap fasteners, VELCRO® and the like.

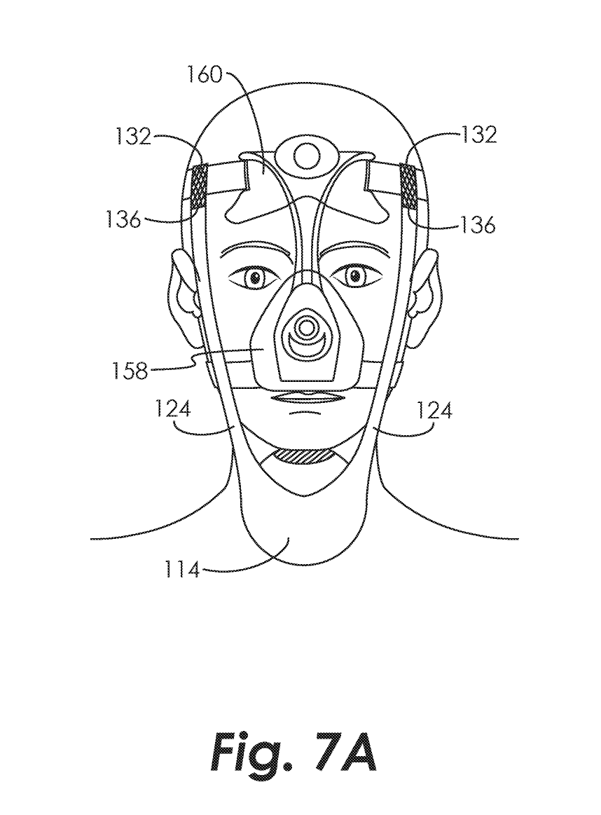

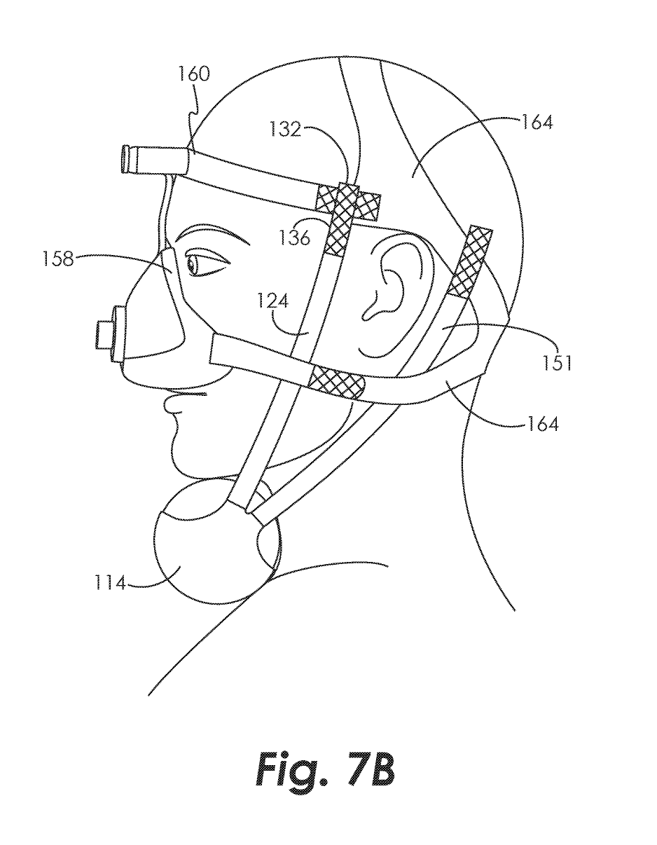

Referring now to FIGS. 7A-7B, an embodiment of a device of the invention is depicted in combination with CPAP nasal mask and headgear. Nasal mask 158 includes a forehead pad 160 and CPAP headgear strap assembly 164. Jaw stop pocket 114, and upwardly extending straps 124 are coupled to the CPAP headgear, at regions 136.

In another aspect of the invention, a device of the invention is used alone to reduce snoring, or in combination with a CPAP system to treat sleep apnea, especially obstructive sleep apnea. For use in reducing snoring, a device including jaw stop and harness assembly is worn without a CPAP system. For treating sleep apnea, a device is used in combination with a CPAP system wherein a user wears the device with a CPAP mask and headgear connected to a CPAP machine to reduce or eliminate the number of nighttime breathing interruptions, improve sleep, and reduce gastric insufflation.

While the form of the device herein described constitutes a preferred embodiment of the invention, it should be understood that the invention is not limited to the precise form of apparatus or device, and that changes may be made therein without departing from the scope of the invention.

### Example 1

### Treating Sleep Apnea

A 30-year old male presents with symptoms of sleep apnea. The patient reports repeated episodes of awakening during the night while gasping for air. He reports feeling tired during the day and sometimes feels bloated in the morning. A medical examination leads to a diagnosis of obstructive sleep apnea (OSA). The patient is prescribed CPAP therapy using a nasal mask. After 1 month of CPAP therapy, the patient reports some reduction in sleep disturbances but feels bloated in the morning. His physician suspects that his mouth has been opening and he has been swallowing air while undergoing CPAP therapy. He recommends use of a mouth-closing device of the present invention to urge the mouth closed and prevent forward head rotation with continued CPAP treatment. The patient begins using a sphere-shaped jaw blocking object in combination with continued CPAP therapy, and after 2 weeks reports substantial improvement in his sleep with fewer air-gasping episodes during the night and no longer feels bloated in the morning.

What is claimed is:

1. A mouth-closing device comprising:
a. a jaw blocking object having a geometric shape other than bent tubular, having a first region on the surface thereof that contacts the underside of a user's chin, and a second region that contacts the user's upper chest clavicular region adapted to prevent the mouth from opening; and
b. a harness assembly adapted to secure the jaw blocking object to the user.

2. A mouth-closing device as in claim 1, wherein the shape of the jaw blocking object is selected from spherical, hemispherical, oblate spherical, prolate spherical, cuboid, cylindrical, and sectioned cylindrical.

3. A mouth-closing device as in claim 2, wherein the jaw blocking object is fabricated from plastic, rubber, silicone, fabric, metal, or leather.

4. A mouth-closing device as in claim 3, wherein the jaw blocking object has a hollow interior.

5. A mouth-closing device as in claim 4, wherein the harness assembly includes a pocket adapted to receive the jaw blocking object, and at least two straps connected to said pocket.

6. A mouth-closing device as in claim 5, further comprising a CPAP mask selected from nasal pillow, nasal mask, nasal prong, full-face mask, hybrid mask, and total face mask.

7. A mouth-closing device as in claim 6 further comprising a CPAP headgear.

8. A mouth-closing device as in claim 7, wherein said at least two straps are adapted to be coupled to the CPAP mask or a headgear.

9. A mouth-closing device for use with a CPAP treatment system, comprising:
a. a spherically-shaped jaw blocking object adapted to be placed under the chin of a user to urge the mouth closed during CPAP treatment said blocking object consisting essentially of first and second contact regions on the surface thereof that make contact with the user;
b. a harness assembly for securing the jaw blocking object under the chin that includes a receiving portion to house the jaw blocking object, said receiving portion having ends, and at least one strap attached to each of said receiving portion ends, said at least one strap having a free end that is adapted to be coupled to a CPAP mask or headgear; and
wherein the device does not contact the user's face above the jaw line.

10. A mouth-closing device as in claim 9 wherein said sphere-shaped jaw blocking object is from 1 to 5 inches in diameter and is fabricated from plastic, silicone, leather, wood, foam, metal, or rubber.

11. A mouth-closing device as in claim 10 wherein said jaw blocking object has a hollow interior.

12. A mouth-closing device as in claim 11 wherein said receiving portion is a pocket that partially or completely encloses the jaw blocking object, and wherein said at least one strap free end is reversibly couplable to the CPAP mask or headgear by a snap, buckle, or VELCRO®.

13. A mouth-closing device as in claim 12 further comprising a CPAP mask.

14. A mouth-closing device as in claim 13 wherein said CPAP mask is selected from nasal pillow, nasal mask, nasal prong, full-face mask, hybrid mask, and total face mask.

15. A method for treating sleep apnea in a patient in need thereof using a CPAP system comprising:
a. placing a CPAP mask and headgear on the patient;
b. securing a jaw blocking object to the CPAP mask and headgear such that the jaw blocking object rests beneath the chin and makes contact with the upper clavicular region of the patient to urge the patient's mouth closed during treatment; and
c. connecting the CPAP mask to a CPAP machine.

16. A method as in claim 15 wherein said jaw blocking object has a shape selected from spherical, hemispherical, oblate spherical, prolate spherical, cuboid, cylindrical, and sectioned cylindrical.

17. A method as in claim 16 wherein said patient is suffering from obstructive sleep apnea.

18. A method as in claim 17 wherein the patient experiences improved sleep and reduced gastric insufflation.
